(19) United States
(12) Patent Application Publication
(10) Pub. No.: US 2026/0232941 A1
(43) Pub. Date: Aug. 13, 2026

(54) FLEXIBLE BREATHING HOSE

(71) Applicant: Loewenstein Medical Technology S.A., Luxembourg (LU)

(72) Inventor: Willy SCHULZE, Roethenbach an der Pegnitz (DE)

(21) Appl. No.: 19/533,483

(22) Filed: Feb. 9, 2026

(30) Foreign Application Priority Data
Feb. 12, 2025 (DE) 10 2025 105 230.1

(51) Int. Cl. A61M 16/08

(52) U.S. Cl. CPC A61M 16/0875; A61M 2205/0216

(57) ABSTRACT

A breathing hose has a continuous wall which delimits an inner lumen, the lumen being designed to conduct respiratory gas. A helix surrounds the wall, on the side facing away from the lumen, in a spiral formation. The wall is made of a first material, and the helix is made of a second material, the helix being connected to the wall. The hose is dimensionally stable and yet flexible.

### CROSS-REFERENCE TO RELATED APPLICATIONS

The present application claims priority under 35 U.S.C. § 119 of German Patent Application No. 10 2025 105 230.1, filed Feb. 12, 2025, the entire disclosure of which is expressly incorporated by reference herein.

### BACKGROUND OF THE INVENTION

### 1. Field of the Invention

The present invention relates to a flexible breathing hose for medical ventilation, in particular for use in CPAP, BiPAP or invasive ventilation systems. The breathing hose has a continuous elastic membrane, which delimits an inner lumen for conducting respiratory gas, and a helix which surrounds the membrane, on the side facing away from the lumen, in a spiral formation.

### 2. Discussion of Background Information

Conventional breathing hoses usually have a pronounced restoring capacity. If such a hose is bent or deflected, a restoring force arises, which seeks to return the hose to its original shape. This restoring force can be transmitted in the form of a tensile load to the ventilation interface, in particular to a breathing mask or a tube.

A tensile load of this kind is often considered uncomfortable by patients and can adversely affect sleep or therapy tolerance. In addition, an increased tensile load can lead to leakage between mask and face, which jeopardizes the success of the therapy.

Flexible breathing hoses are known from the prior art, but they have either a comparatively high degree of stiffness or high extensibility. For example, DE 10 2024 110 733 A1 discloses a hose with a spring rate of at least 25 N/m. EP 2 438 953 B1 describes a hose that is extensible to 40% to 400% of its original length. However, such hoses have either a high restoring force or an insufficient dimensional stability, especially under purely gravitational loads.

Against this background, it would be advantageous to have available a breathing hose which has a low restoring force and thereby exerts a reduced tensile load on the ventilation interface, but which maintains sufficient dimensional stability and kink stability.

### SUMMARY OF THE INVENTION

In a first aspect, the invention provides a breathing hose which has a continuous elastic membrane, which delimits an inner lumen for conducting respiratory gas. A helix surrounds the membrane, on the side facing away from the lumen, in a spiral formation. The membrane consists of a first polymer material, while the helix consists of a second polymer material different than the first one. The helix is positively connected and/or cohesively bonded to the membrane.

In one embodiment, the restoring force of the breathing hose, measured according to the test method set forth below, is at most 0.6 N, preferably at most 0.4 N, particularly preferably at most 0.2 N.

In one embodiment of the hose, in a bending test under the exclusive influence of the inherent weight of the hose, the horizontal deflection of the hose initially increases nonlinearly, with increasing vertical deflection, and then reaches a substantially constant value.

In the context of the present application, this behavior is evaluated as particularly favorable draping behavior, since the hose already reaches its maximum flexural deformability even at a small vertical deflection and does not show any significant additional horizontal deflection under further loading.

### Draping Behavior and Restoring Force

In preferred embodiments, starting from a vertical deflection of at most 150 mm, the horizontal deflection of the hose changes by less than 10% upon a further increase of the vertical deflection.

With a vertical deflection of 100 mm, the horizontal deflection is preferably at most 100 mm. The described draping behavior can be mathematically described approximately by an asymptotic function. This mathematical description serves in particular to explain the curve profile.

### Temperature Behavior

In preferred embodiments, the restoring force of the hose at a temperature of 20° C. and at a temperature of 40° C. differs by less than 20%. This is particularly advantageous for ventilation systems with temperature-controlled and humidified respiratory gas.

### Materials and Geometry

The membrane consists preferably of a thermoplastic elastomer (TPE), TPE-S or silicone.

The wall thickness of the membrane is preferably in the range of 0.1 mm to 0.3 mm.

The helix preferably consists of TPE, TPE-S or silicone and has a diameter of 0.4 mm to 2.5 mm.

The helix gives the hose stability against kinking, and therefore, in the event of bending with a radius of 20 mm, the lumen does not collapse.

Membrane and helix can preferably be manufactured by multi-component injection molding or by overmolding.

The drapeability of the hose can be described as an asymptotic function, such as

$${o}^{} f ( x ) = a - b \cdot {e}^{- c \cdot x}$$

The horizontal extension (Horizontal Distance Out) of the hose under vertical loading with the inherent weight of the hose (Vertical Distance Down) is approximately the same (up to +15% greater) as the (hose length of the) applied vertical load (Vertical Distance Down), or less.

The horizontal extension of the hose under vertical loading with the inherent weight of the hose, at least for lower loads below 100 mm, is preferably smaller than the length of the applied vertical load.

Preferably, in terms of drapeability and restoring force, the hose behaves at 20° C. very similarly to the way it behaves at 40° C.

The breathing hose according to the invention has a continuous membrane, which is preferably made of TPE or TPE-S or silicone.

In one embodiment, the breathing hose according to the invention has a continuous membrane whose wall thickness is 0.1 mm to 2 mm, preferably 0.15 mm.

Preferably, the helix is made of TPE or polyolefins, and the diameter of the helix is in the range of 0.4 to 2.5 mm, preferably 2 mm.

The restoring capacity changes very little, especially in a temperature range of 20 to 40° C. This in turn affords a significant advantage in respiratory therapies in which the respiratory gas is administered in a temperature-controlled form. In practise, respiratory gas temperatures of up to 40° C. and 100% humidity are usually reached, depending on the type of therapy.

The advantages described above are mainly obtained through the material-specific properties, in particular of the so-called membrane (which encloses the inner lumen). However, the flexibility or restoring capacity differs significantly from all of the known, geometrically comparable hoses on the market.

### BRIEF DESCRIPTION OF THE DRAWINGS

Embodiments of the invention are described in the following with reference to the accompanying drawings. Neither the description nor the drawings should be understood as limiting the scope of the invention. In the drawings:

FIG. 1 shows a breathing hose with a continuous membrane, which delimits an inner lumen, and with a helix.

FIG. 2 shows a measuring device for determining the restoring force of the breathing hose.

FIGS. 3 and 4 show measurement results for the restoring force and the drapeability of different hose types.

FIGS. 5a to 5d illustrate the measurement method for determining the horizontal deflection of a hose with increasing vertical deflection under the influence of its inherent weight.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

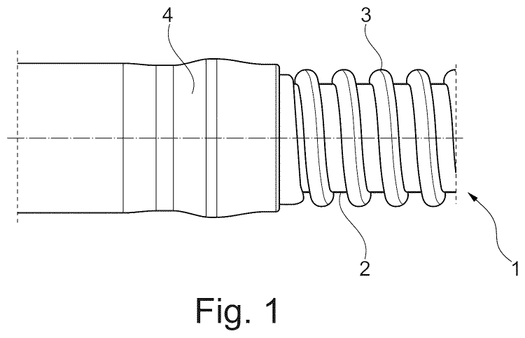

FIG. 1 shows a breathing hose (1) with a continuous membrane (2) which delimits an inner lumen. The lumen is designed to conduct respiratory gas. A helix (3) surrounds the membrane (2), on the side facing away from the lumen, in a spiral formation. The membrane (2) is made of a first material and the helix (3) is made of a second material. The internal diameter is 15-22 mm. The restoring force of the hose is preferably below 0.6 N and particularly preferably below 0.4 N and very particularly preferably 0.2 N.

At one end, the hose can have a sleeve which serves for attachment to a ventilator. The other end (not shown) of the hose can have an adapter which serves for the coupling of a mask.

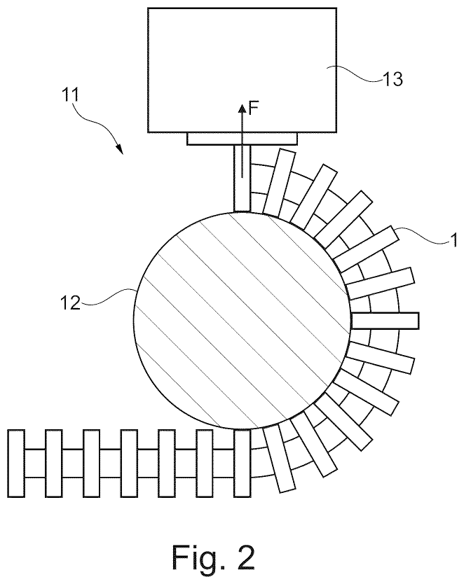

FIG. 2 shows a breathing hose (1) according to FIG. 1, which is placed into a measuring device (11) for determining the restoring force in Newton (N).

### The Restoring Force of the Hose is Determined as Follows:

The hose (1) is placed, with a suitable length, horizontally onto a round body (12) (a circular or oval device) with a diameter of, for example, 90 mm. In this case, the hose has to lie approximately on half of the circumference of the round body. The part of the hose not located on the round body lies straight and unloaded on a flat surface. A load cell (5) is mounted exactly centrally over the round body. The upper end of the hose then presses against the load cell (5) due to the restoring capacity of the hose. The resulting force F is the restoring force of the hose.

A low restoring force of a hose means that the hose will provide less resistance when bent or deformed and will return to its original shape with only little force.

The importance of a low restoring force lies in the increased flexibility. The hose is softer and can be more easily bent without creating strong resistance. This can be particularly advantageous when freedom of movement is required.

The restoring force and drapeability of various hoses of diameter Ø15 mm were determined.

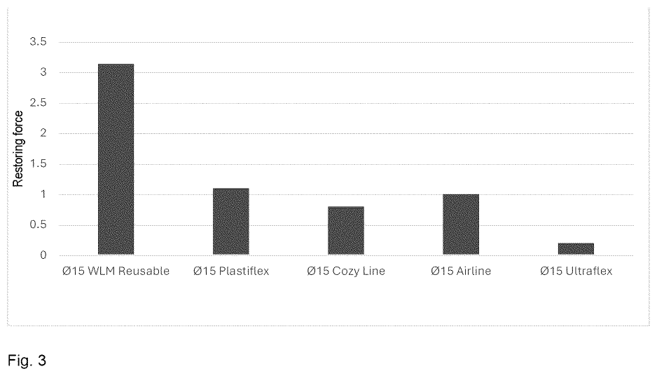

FIG. 3 shows the respective restoring force in Newton (N) for different hoses.

### Analysis of Values:

WLM Reusable has the highest restoring force with 3.14 N.
UltraFlex (UF) has the lowest restoring force with 0.2 N.
The other hoses are in between:
   Plastiflex: 1.1 N
   Cozy Line: 0.8 N
   Airline: 1.0 N

The diagram clearly illustrates the differences, with the Ø15 WLM Reusable hose highest and the Ø15 UF hose lowest.

The restoring force of a hose depends on several factors, including the material, diameter, wall thickness, and the deformation (e.g. bending or stretching) to which the hose is subjected.

To calculate the restoring force F of a hose, a simplified approach can be used applying Hooke's law, if the deformation is elastic:

F=k·ΔxF=k\cdot\Delta xF=k·Δx

### Here:

F is the restoring force (in Newton),
k is the spring constant or stiffness of the hose (in N/m),
Δx\Delta xΔx is the deformation (lengthening or shortening) of the hose (in meters).

### The Spring Constant k Depends on:

the material modulus (elastic modulus) of the plastic,
the geometry of the hose (diameter, wall thickness).

Specific material data and the exact geometry of the hose are relevant for a more precise calculation. In practise, the restoring force can also be determined by experimental measurement, i.e. by deforming the hose by a known length and measuring the required force.

Less tensile force on the patient: In medical uses, e.g. in breathing hoses, this means that the hose pulls to a lesser extent on the mask or the tube, thereby improving patient comfort. Easy handling: Hoses with a low restoring force are often more convenient to position, and they adapt better to different lying positions or sitting positions. Loss of stability possible: However, a very low restoring force can have the effect that the hose kinks slightly or does not hold its shape well, which could adversely affect the flow of air. In summary: A hose with a low restoring force is more flexible and more comfortable, but possibly less stable. In the present case, the stability is also determined via the helix.

### Importance of a Low Restoring Force:

1. Increased flexibility: The hose is softer and can be more easily bent without creating strong resistance. This can be particularly advantageous when freedom of movement is required.
2. Less tensile force on the patient: In medical uses, e.g. in breathing hoses, this means that the hose pulls to a lesser extent on the mask or the tube, thereby improving patient comfort.
3. Easy handling: Hoses with a low restoring force are often more convenient to position, and they adapt better to different lying positions or sitting positions.
4. Loss of stability possible: However, a very low restoring force can have the effect that the hose kinks slightly or does not hold its shape well, which could adversely affect the flow of air.

The restoring force of a hose has a direct influence on its flexibility, stability and handling.
1. High restoring force (e.g. WLM Reusable-3.14 N)
   Suitable for uses involving high mechanical loads
Disadvantages:
   Less flexible, more difficult to position
   May exert greater tension on the ventilation interface (e.g. mask or tube)
2. Medium restoring force (e.g. Plastiflex-1.1 N, Airline-1.0 N, Cozy Line-0.8 N)
   Good compromise between flexibility and stability
Disadvantages:
   May still bend slightly in some situations
3. Very low restoring force (e.g. UF-0.2 N)
Advantages:
   Very flexible, easily adapts to movements
   Minimum tension exerted on the patient, high level of comfort
   Ideal for mobile or sensitive patients

The UF hose is the most flexible. It is suitable in particular, for uses where freedom of movement and comfort are critical.

The drapeability of a hose refers to the ability of the hose to adapt to a particular shape or surface without exerting strong resistance forces. The drapeability of a hose is determined by means of a bending test under the exclusive influence of the inherent weight of the hose. The drapeability of a hose thus describes how flexible or stiff the hose is and how well it is able to bend under its own weight or by external action without returning to its original shape.

Factors that Affect Drapeability:
1. Material properties:
   Modulus of elasticity (elastic modulus): A low elastic modulus results in greater flexibility and better drapeability.
   Density: Heavier materials can drape better under their own weight.
2. Wall thickness:
   Thin-walled hoses are more drapeable than thick-walled hoses because they are more easily bendable.
3. Diameter:
   Hoses with a small diameter are usually more flexible and drapeable than those with a large diameter.
4. Temperature:
   Many plastics become softer at higher temperatures, which improves drapeability.
5. Plasticizer content:
   Plastics with a higher plasticizer content are more flexible and have better drapeability.

### Example of Use:

A hose with high drapeability is well suited for uses where the hose has to wrap around corners or irregular shapes, e.g. in medical technology or when installed in confined spaces.

The quantitative determination of the flexibility/restoring capacity is carried out by means of a drapeability test. The drapeability of a hose is determined by means of a bending test under the exclusive influence of the inherent weight of the hose, wherein the horizontal deflection of the hose and/or the vertical deflection are determined.

The horizontal extension (Sample Horizontal Distance Out in mm) of the hoses is determined according to the vertical load under the inherent weight of the respective hose.

TABLE 1
Drapeability

| | Vertical Distance Down in mm - | | | | | | | | | | | |
|---|---|---|---|---|---|---|---|---|---|---|---|---|
| | Sample Horizontal Distance Out in mm | | | | | | | | | | | |
| Hose marking mm | 25 | 50 | 75 | 100 | 125 | 150 | 175 | 200 | 225 | 250 | 275 | 300 |
| UF Ø15 | | | | | | | | | | | | |
| 300 | 25 | 30 | 38 | 43 | 46 | 48 | 48 | 48 | 48 | 48 | 48 | 48 |
| 400 | 18 | 26 | 32 | 36 | 38 | 40 | 40 | 40 | 40 | 40 | 40 | 40 |
| 600 | 18 | 24 | 27 | 30 | 32 | 32 | 32 | 32 | 32 | 32 | 32 | 32 |
| WLM Standard Ø15 | | | | | | | | | | | | |
| 300 | 156 | 250 | | | | | | | | | | |
| 400 | 85 | 137 | 182 | 220 | 256 | 295 | | | | | | |
| 600 | 36 | 60 | 80 | 95 | 110 | 125 | 136 | 145 | 155 | 165 | 172 | 180 |
| WLM Reuse Ø15 | | | | | | | | | | | | |
| 300 | 70 | 95 | 120 | 160 | 190 | 220 | | | | | | |
| 400 | 70 | 85 | 100 | 110 | 120 | 140 | 145 | 155 | 164 | 175 | 185 | 195 |
| 600 | 55 | 80 | 95 | 110 | 120 | 130 | 140 | 145 | 145 | 145 | 145 | 145 |
| Plastiflex Ø15 | | | | | | | | | | | | |
| 300 | 40 | 55 | 65 | 75 | 85 | 95 | 110 | | | | | |
| 400 | 92 | 125 | 155 | 187 | 210 | 230 | 88 | 90 | 95 | 100 | 105 | |
| 600 | 55 | 72 | 90 | 102 | 115 | 120 | 125 | 132 | 132 | 132 | 132 | 132 |
| Cozy Line Ø15 | | | | | | | | | | | | |
| 300 | 50 | 73 | 95 | 118 | 143 | 37 | 37 | 37 | 37 | 37 | 37 | 37 |
| 400 | 45 | 63 | 78 | 88 | 95 | 100 | 60 | 65 | 70 | 85 | 90 | 100 |
| 600 | 30 | 38 | 43 | 48 | 50 | 50 | | | | | | |

The ratio of “horizontal deformation” to “vertical deformation” indicates a measure of flexibility.

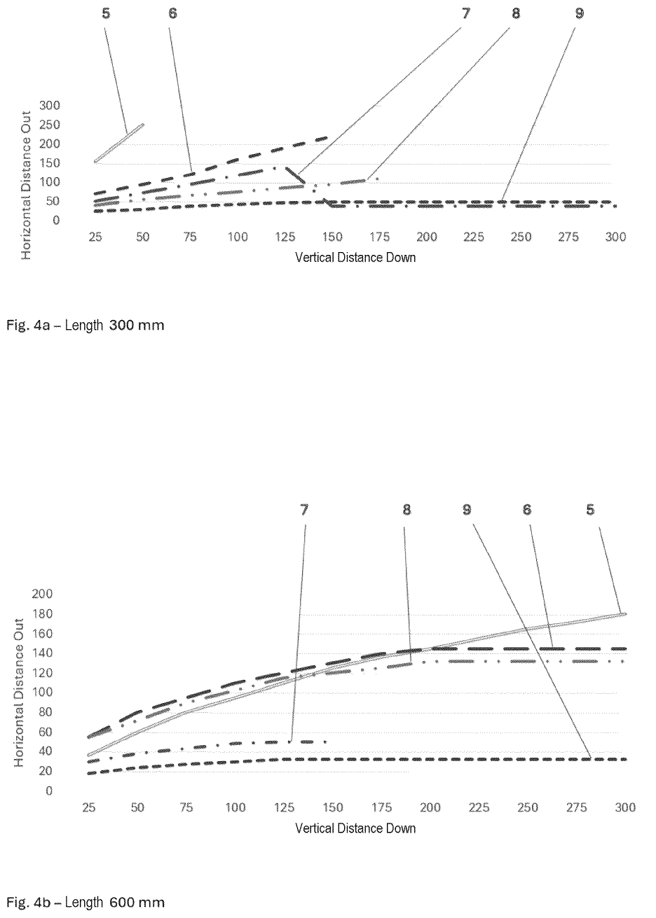

The measured values in Table 1 are plotted in FIGS. 4a and 4b.

### FIG. 4 Drapeability

The figures show measurement results of hoses. The horizontal extension (Sample Horizontal Distance Out in mm) of the hoses is measured according to the vertical load (Vertical Distance Down in mm) for different hose lengths (300 mm, 600 mm).

The measured values are from five different types of hoses with a diameter of Ø15 mm:
(9) UF
WLM Standard (5)
WLM Reuse (6)
Plastiflex (8)
Cozy Line (7)

### Analysis of the Measurement Results

1. UF (9)
   Shows the lowest horizontal extension under load. The horizontal extension (horizontal distance) even comes to a stop under an increasing load and does not grow any further.
   Remains very compact and changes its shape only minimally.
   Low restoring force→very flexible

For the hose length 300 mm, the horizontal extension of the hose, under a vertical load with the inherent weight of the hose (gravity), gradually increases from 25 mm to 48 mm at 150 mm (Vertical Distance Down)

For the hose length 600 mm, the horizontal extension of the hose, under a vertical load with the inherent weight of the hose (gravity), gradually increases from 18 mm to 32 mm at 125 mm (Vertical Distance Down)

However, the horizontal extension of the hose under a vertical load with the inherent weight of the hose (Vertical Distance Down) is not greater than the (length of the) applied vertical load (Vertical Distance Down).

The horizontal extension of the hose, under a vertical load with the inherent weight of the hose (Vertical Distance Down), is always approximately the same (up to +15%) as the (length of the) applied vertical load (Vertical Distance Down) or less.
2. WLM Standard (5)
   Has a very high horizontal extension under load.
   Especially at 400 mm and 600 mm length, the hose reaches values of up to 295 mm.
   High stability, but less flexible.
3. WLM Reuse (6)
   Medium to high horizontal extension, especially with longer hose segments (600 mm to 195 mm).
   Compromise between stability and flexibility.
4. Plastiflex (8)
   Shows a medium horizontal extension, but the values are inconsistent (e.g. 400 mm length: jump from 230 mm to 88 mm).
   Has different deformation properties depending on the load.
5. Cozy Line (7)
   Relatively low horizontal extension (max. 143 mm at 300 mm length).
   Not as flexible as other models.

### Technical Interpretation of the Differences

Hoses with high horizontal extension (e.g. WLM Standard Ø15) are less flexible but very stable. They are suitable for uses where dimensional stability is critical.
Hoses with low horizontal extension (e.g. UF Ø15) are very flexible and adapt well to movements
Medium values (e.g. WLM Reuse Ø15, Plastiflex Ø15) offer a compromise of stability and flexibility

The diagrams show that the horizontal extension (Horizontal Distance Out) of the Cozy Line and Plastiflex hoses suddenly decreases sharply from a vertical load of 150 mm.

Possible Reasons for this Drop:

The reduction of the horizontal deflection is mainly due to the fact that the hose has a limited bending radius. The sudden drop is due to the fact that the hose has reached its maximum measurable deformation at 150 mm.

The drop in horizontal extension shows that Cozy Line and Plastiflex lose their structural integrity under increasing vertical loads. This means in practise that, although they are very flexible, when subjected to loads they rapidly yield, bend or become plastically deformed. They are therefore better suited for uses where maximum flexibility is required, but not for uses where a dimensionally stable hose is needed.

Advantages of the Behavior of the UF Hose in this Area:

In the tables and diagrams, the UF Ø15 hose shows a very small change in horizontal extension, even with an increasing vertical load.

The great advantage is that the UF hose already has its maximum flexibility (flexural deformability) at a very small vertical deflection. The other hoses only reach this point at significantly higher vertical loads or do not reach it at all due to being too stiff. The table of values for UF at 300 mm shows a function where the values first increase and then stabilize at 48.

The values initially rise nonlinearly and then reach a plateau. A possible function would be an asymptotic function such as:

$$f ( x ) = a - b \cdot {e}^{- c \cdot xf}$$

By regression, the function for 300 mm for UF can be determined, for example, as:

$$f ( x ) = 51.4 - 39.6 \cdot {e}^{- 0.0146 \cdot x}$$

For example, the calculated function to describe the values for 600 mm for UF is:

$$f ( x ) = 33.29 - 25.22 \cdot {e}^{- 0.0199 \cdot x}$$

This function shows that the values asymptotically approach the maximum value of approximately 32.

This means:
1. High dimensional stability.
   The hose remains constant in its shape, even if it is loaded.
   There is no sudden collapse or kinking as with Cozy Line or Plastiflex.
2. Prevents obstructions to air flow
   Since the hose does not uncontrollably deform or collapse, the air flow remains stable.
   This is particularly important for breathing hoses, since a constant air supply has to be ensured.
3. Increased longevity and reduced material fatigue
   UF does not lose its elastic properties so quickly under loads.
   This indicates that the material has a high restoring force and an intelligent combination of materials is used.
4. Good balance between flexibility and stability
   While Cozy Line and Plastiflex are too soft and unstable, UF appears to have found a good balance.
   This allows comfortable handling without the disadvantages of an unstable hose.
      Possible Materials or Material Combinations for this Advantage
1. Silicone with reinforced fabric
   Silicone is flexible, temperature resistant and durable.
   Internal reinforcement by textile fibers or a thin wire mesh could provide additional stability.
2. Thermoplastic elastomers (TPE) optionally with polyurethane (PU) coating
   TPE ensures flexibility and elasticity, so that the hose remains easily bendable.
   PU provides some stiffness and resistance to buckling.
3. Multi-layer construction with a hard outer layer and a soft inner layer
   Outer layer made of polypropylene (PP) or polycarbonate (PC) for dimensional stability.
   Inner layer made of flexible silicone or latex for pleasant flexibility.
   The hose thus remains flexible but stable.
4. Helix for strengthening
   A hose with an integrated or attached spiral made of nylon, silicone or a TPE or TPE-S can help to maintain the shape.

The UF hose has a combination of materials that gives it both flexibility and dimensional stability. A flexible membrane and a stiff helix, permanently connected to each other, are an aspect that contributes to the properties.

For example, the optimal mixture of an elastic material (silicone or TPE or TPE-S) for the membrane and of a reinforcing layer, in the form of a fabric or a helix or a coating that surrounds the membrane, is responsible for this. In the case of a helix, the latter has stiffening properties and is preferably made of a material different than the membrane. This makes the hose ideal for uses in which the hose needs to adapt well but must not collapse. The materials can preferably be processed by injection molding, even in multi-component injection molding applications. The materials are preferably skin-friendly and suitable for medical uses.

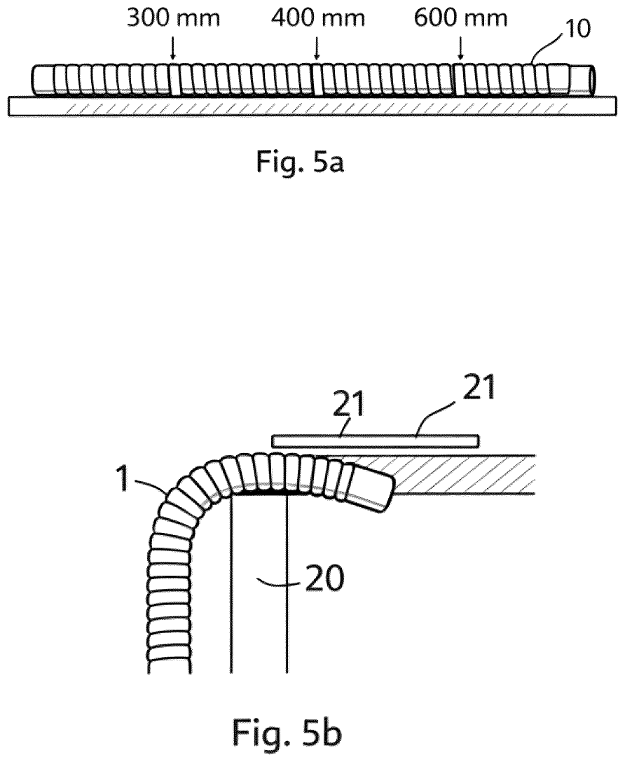

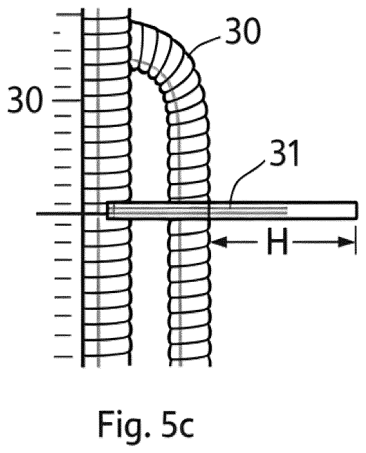

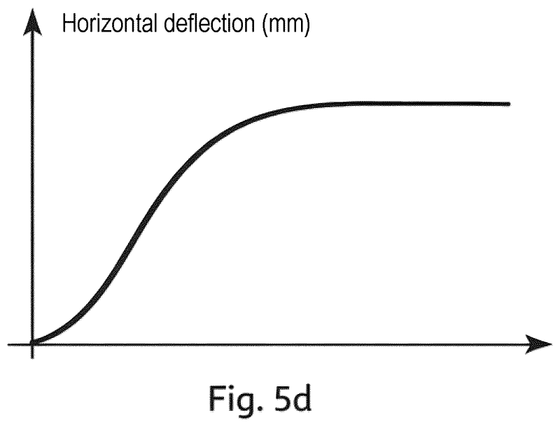

FIGS. 5a-5d illustrate the measurement method for drapeability

There is no standardized measure for drapeability such as there is for flexibility, but it can be determined qualitatively by observation or experimentally by bending tests and stress tests.

The drapeability of a material can be tested in various ways. Here are some common methods:
1. Drape test: A circular sample of material is placed onto a plate and hangs down under gravity. The drapeability is measured by the draping coefficient, which represents the ratio of the projected surface to the original surface.
2. DIN EN ISO 21765: This international standard describes a standardized method for determination of the deformability of textiles by forced mechanical distension. It enables comparable and reproducible measured values for drapeability.

In the present case, it was attempted to determine drapeability in accordance with the standard DIN EN ISO 21765. However, DIN EN ISO 21765 is applicable to textiles that are considerably more flexible. Therefore, a modified measurement method was applied.

When testing drapeability, only the weight force (F=m×g) acts as bending force on the hose. The resulting ratio of “horizontal deformation” to “vertical deformation” then indicates a measure of the drapeability.

The hose is held with a length of 300 mm over the edge of a straight surface arranged at 90°, so that the hose, on account of gravity, tilts downward with a certain radius. The drapeability is determined by determining the horizontal point at which the hose intersects with a straight ruler at the vertical height of −25 mm. This procedure is repeated in vertical 25 mm steps. (−50 mm, −75 mm, −100 mm, until a vertical length of −300 mm is reached or until the hose no longer meets the horizontal ruler. The correspondingly determined horizontal intersection points must be displayed in an X/Y diagram depending on the vertical reference dimension.

The drapeability of the breathing hose is measured, for example, by guiding a ruler along the vertical measurement scale at a distance of 25 mm until either 300 mm is reached or the hose no longer crosses the ruler. The point at which the hose crosses the ruler is noted as a measuring point. This procedure is repeated for the 400 and 600 mm marks

The measurement method described below is used to quantitatively determine the bending properties of a flexible breathing hose, in particular the horizontal deflection of the hose as a function of an increasing vertical deflection, which is caused exclusively by the inherent weight of the hose (drapeability).

FIG. 5a shows the preparation of the hose sample for defining the test lengths and initial geometry.

### Illustration:

Longitudinal view of a straight breathing hose (1)
Markings at 300 mm, 400 mm and 600 mm
Illustration of a longitudinal measuring device or channel

### Reference Signs:

(1) Breathing hose
(10) Markings of the test lengths

A breathing hose to be tested is provided in a straight, unloaded starting position. In the section to be tested, the hose has no kinks, permanent deformations or preliminary damage. Defined test lengths are marked on the hose, for example:
300 mm,
400 mm and
600 mm,
   measured in each case from one end of the hose along the longitudinal axis of the hose.

FIG. 5b shows the positioning of the hose at the holding edge in order to illustrate the clamping without clamps and the purely gravitational loading.

### Illustration:

Hose (1) lies with a marking on a horizontal edge (20)
Free-hanging hose section below the edge
Guide element (21)—for example a pipe—above the edge to prevent upward bending

### Reference Signs:

(20) Holding edge
(21) Guide element

FIG. 5c shows the measurement of the horizontal deflection for defining the horizontal deflection at a certain vertical deflection (drapeability).

### Illustration:

Vertical scale (30) next to the hose
Horizontal measuring ruler (31) at a defined vertical height
Intersection point between hose and horizontal ruler
Drawn horizontal measuring distance (H)

### Reference Signs:

(30) Vertical scale
(31) Horizontal measuring ruler
(H) Horizontal deflection

### i) Clamping and Initial Position

The hose is positioned with the respective marking on a horizontal edge of a holding device, so that the hose section hangs freely down from the marking.

The hose lies with a form fit in the region of the edge, without being clamped, so that no additional holding forces are effective except for the inherent friction. Above the edge, the hose is secured by a guide element (21) or a support against unwanted upward bending.

The free-hanging hose section is loaded exclusively by gravity and adopts a stable resting position.

### ii) Determination of Vertical Deflection

A vertical scale is arranged parallel to the hanging hose length, the zero point of the vertical scale being at the height of the hose support edge.

Starting from this edge, the vertical deflection is measured downward in discrete steps, preferably in 25 mm increments, starting at 25 mm and up to a maximum vertical deflection of, for example, 300 mm or until the point at which the hose no longer has any measurable horizontal deflection.

iii) Determination of Horizontal Deflection

At each specified vertical measurement height, a horizontal measuring ruler or a horizontal measuring plane is positioned such that it:
is oriented perpendicular to the vertical scale and
is located at the appropriate vertical height without touching the hose.

The horizontal deflection is defined as the horizontal distance between the vertical reference plane (through the hose support edge) and the intersection point of the hose with the horizontal measurement plane. This value is collected and documented for each vertical deflection.

### Repetition for Different Hose Lengths

Steps i) to iii) are repeated for each previously marked hose length (e.g. 300 mm, 400 mm, 600 mm) in order to determine the bending behavior of the hose as a function of the effective hose length.

FIG. 5d shows the results in diagram form

### Illustration:

Diagram with:
   X-axis: Vertical deflection (mm)
   Y-axis: Horizontal deflection (mm)
Nonlinearly ascending curve with subsequent plateau

Demonstrating the characteristic bending behavior of the hose.

The measured values of the horizontal deflection are displayed in a coordinate system depending on the respective vertical deflection, where:
the vertical deflection is plotted on the abscissa and
the horizontal deflection is plotted on the ordinate.

The resulting curve characterizes the draping and bending behavior of the hose.

A nonlinear increase in the horizontal deflection with subsequent transition to a region with substantially constant horizontal deflection is regarded as an indication of a particularly favorable interplay between flexibility and dimensional stability.

What is claimed is:

1. A breathing hose, wherein the hose comprises
a continuous elastic membrane, which delimits an inner lumen for conducting respiratory gas,
a helix, which surrounds the membrane, on a side facing away from the lumen, in a spiral formation,
wherein
the membrane consists of a first polymer material and the helix consists of a second polymer material different from the first polymer material,
the helix is positively connected and/or materially bonded to the membrane,
and wherein
a restoring force of the breathing hose is at most 0.6 N and, in a bending test under exclusive influence of an inherent weight of the hose, a horizontal deflection of the hose initially increases nonlinearly, with increasing vertical deflection, and then reaches a substantially constant value.

2. The breathing hose of claim 1, wherein in the bending test a horizontal deflection, starting from a vertical deflection of at most 150 mm, changes by less than 10% upon a further increase of the vertical deflection.

3. The breathing hose of claim 1, wherein the restoring force is at most 0.4 N.

4. The breathing hose of claim 1, wherein the restoring force is at most 0.2 N.

5. The breathing hose of claim 1, wherein the horizontal deflection of the hose, at a vertical deflection of 100 mm, is at most 100 mm.

6. The breathing hose of claim 1, wherein the restoring force of the hose at a temperature of 20° C. and at a temperature of 40° C. differs by less than 20%.

7. The breathing hose of claim 1, wherein the membrane is made of a thermoplastic elastomer (TPE), TPE-S or silicone.

8. The breathing hose of claim 1, wherein a wall thickness of the membrane ranges from 0.1 mm to 0.3 mm.

9. The breathing hose of claim 1, wherein the helix has a diameter of from 0.4 mm to 2.5 mm.

10. The breathing hose of claim 1, wherein the helix is made of TPE, TPE-S or silicone.

11. The breathing hose of claim 1, wherein the helix imparts to the hose stability against kinking, wherein a bending of the hose with a radius of 20 mm does not cause the lumen to collapse.

12. The breathing hose of claim 1, wherein the membrane and the helix are manufactured by multi-component injection molding or by overmolding.

13. The breathing hose of claim 1, wherein the hose is configured for use in a CPAP, BiPAP or invasive ventilation therapy, is lined with a continuous membrane which delimits an inner lumen, the lumen being configured to conduct respiratory gas, and a helix surrounds the membrane, on the side facing away from the lumen, in a spiral formation, wherein the internal diameter is 15-22 mm and the restoring force of the hose is less than 0.6 N, and wherein the hose has, at one end, a sleeve which serves for attachment to a ventilator and, at the other end, an adapter which serves for the coupling to a breathing mask.
